(19) United States
(12) Patent Application Publication
(10) Pub. No.: US 2024/0189116 A1
(43) Pub. Date: Jun. 13, 2024

(54) IMPLANTABLE OESOPHAGUS PROSTHESIS

(71) Applicant: Kerstin-Evelyne VOIGT, Argeles sur mer (FR)

(72) Inventor: Kerstin-Evelyne VOIGT, Argeles sur mer (FR)

(21) Appl. No.: 18/554,690

(22) PCT Filed: Apr. 20, 2022
(86) PCT No.: PCT/EP2022/060355

§ 371 Date: Oct. 10, 2023

(30) Foreign Application Priority Data
Apr. 20, 2021 (BE) 2021/5309

(51) Int. Cl. A61F 2/48; A61F 2/04

(52) U.S. Cl. CPC A61F 2/484; A61F 2/482; A61F 2002/044; A61F 2250/0013

(57) ABSTRACT

Implantable oesophageal prosthesis comprising a feeding tube, which feeding tube is at least partially surrounded by a housing in which a pressurising member is applied, which pressurising member comprises a pressure application element provided for generating a peristaltic pressure against an outer wall of the feeding tube, which pressurising member is applied in such a manner as to be movable along the feeding tube over a predetermined distance starting from an initial position, which pressure application element is provided for generating the peristaltic pressure continuously during its movement along the feeding tube in a first direction extending towards the stomach of a body in which the prosthesis is to be implanted, said pressurising member comprises a return member provided for applying a return force extending in a second direction, opposite to the first direction, in order to return the pressure application element towards its initial position.

The present invention relates to an Implantable oesophageal prosthesis comprising a feeding tube, which feeding tube is at least partially surrounded by a housing in which a pressurising member is applied, which pressurising member comprises a pressure application element provided for generating a peristaltic pressure against an outer wall of the feeding tube.

Such an implantable oesophagus prosthesis is known from WO2019/241414 and is used for replacing a natural oesophagus in a body of a person. The known implantable oesophagus prosthesis comprises a tube, made of for example silicone for bio-medical applications. The prosthesis also comprises a plurality of houses in which a pressurising member is each time lodged, which pressurising member comprises a pressure application element provided for generating a peristaltic pressure against an outer wall of the feeding tube and thus contribute to the displacement of food in the feeding tube.

A drawback of the known implantable oesophagus prosthesis is that due to the use of a plurality of houses it is not possible to apply a continuous pressure over the length of the implanted oesophageal. Indeed the pressure is applied at a place where the housing is situated but not on the part between two subsequent houses. This could lead to a situation where the introduced food might get blocked into the implanted oesophagus prosthesis between two subsequent houses and thus harm the person in which the prosthesis is implanted. The reliability of the known implantable oesophagus prosthesis is thus not sufficient for application to a human body.

It is an object of the present invention to realise a more reliable implantable oesophagus prosthesis.

To this purpose the implantable oesophagus prosthesis according to the present invention is characterised in that the pressurising member is applied in such a manner as to be movable along the feeding tube over a predetermined distance starting from an initial position, which pressure application element is provided for generating the peristaltic pressure continuously during its movement along the feeding tube in a first direction extending towards the stomach of a body in which the prosthesis is to be implanted, said pressurising member comprises a return member provided for applying a return force extending in a second direction, opposite to the first direction, in order to return the pressure application element towards its initial position. By generating a continuous peristaltic pressure against the outer wall of the feeding tube during its movement along the feeding tube in a first direction extending towards a stomach of a human body in which the prosthesis is to be implanted, care is taken that a continuous pressure is applied against the food present in the feeding tube and to be transported to the stomach. This enables to transport the food through the oesophagus prosthesis in a reliable manner, thereby considerably reducing the risk that the food gets stuck in the oesophagus prosthesis.

It will be noted that the patent application US2018/0125633 describes a device which can be implanted in the natural oesophageal of a living body and which can be moved in this oesophageal and respond to forces produced by peristaltic movements of the natural oesophageal. However, the device described in US2018/0125633 cannot be combined with the prosthesis described in WO2019/241414. Indeed, the device according to US2018/0125633 is placed inside the natural oesophageal, whereas in WO2019/241414 the pressurising member is placed outside a prosthesis which replaces the natural oesophageal. A combination of the teaching of the two patent applications is thus technically speaking impossible.

A first preferred embodiment of an implantable oesophagus prosthesis according to the invention is characterised in that the pressure application element is provided for generating the continuous peristaltic pressure by using a hydraulic fluid or a gas under pressure. The use of a hydraulic fluid or a gas under pressure offers an appropriate solution for applying the continuous peristaltic pressure against an outer wall of the tube implanted in a human body.

Preferably the first preferred embodiment of an implantable oesophagus prosthesis according to the invention is characterised in that the pressure application element comprises a fluid input and a fluid output, which the pressure application element comprises an internal wall configured for at least partially enveloping the feeding tube and provided with a first series of output gates and at least a second series of input gates, the input gates being applied, considered in said first direction, under the output gates, the output gates, respectively the input gates, being connected to said fluid input, respectively to said fluid output, the fluid input being connected to an pump output provided for furnishing the hydraulic fluid at a pressure of at least 2 bars. The outlet gates enable a direct output of the fluid thereby creating the continuous peristaltic pressure against the outer wall of the tube. The inlet gates on their turn enable to collect back the fluid exhausted by the outlet gates and thus to obtain a closed fluid circuit.

Preferably the first preferred embodiment of an implantable oesophagus prosthesis according to the invention is further characterised in that the return member is formed by a spring lodged at the inside of said housing. This enables a fast and reliable return of the cylindrical body to the initial position.

A second preferred embodiment of an implantable oesophagus prosthesis according to the invention is characterised in that the pressure application element comprises a set of compression members provided for exerting a back-and-forth movement with respect to the feeding tube. The compression members offer a mechanical reliable solution.

Preferably the second preferred embodiment of an implantable oesophagus prosthesis according to the invention is characterised in that the pressure application element comprises a pusher and a compressor mounted on a driving member provided for generating said movement along the tube, said compressor being mounted downstream of said pusher, said compression members being part of said compressor, said pusher being provided for imposing a movement on said compression members in order to enable said back and forth movement. The use of a pusher and a compressor enable the back-and-forth movement. The use of a pusher and a compressor enables to easily in function of the subsequent time sequences manage the back and forth movement along the tube.

Preferably the second preferred embodiment of an implantable oesophagus prosthesis according to the invention is further characterised in that the compression elements are each formed by a roller rotatably applied on an axis. The use of rotatably applied rollers enables to reduce the friction between the compression members and the tube thereby reducing the power consumption and increase the lifetime of the prosthesis.

The invention will now be described in more details by referring to the annexed drawings. In the drawings:

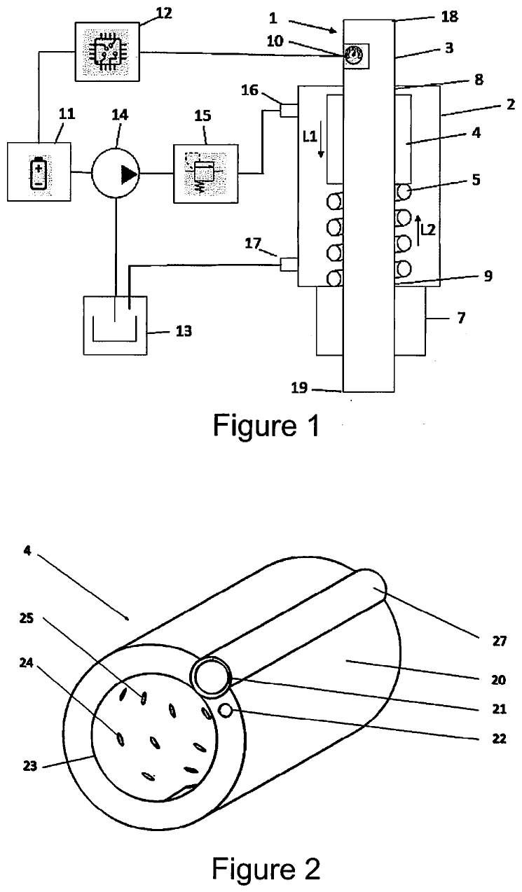

FIG. 1 shows a first preferred embodiment of an implantable oesophagus prosthesis according to the invention;

FIG. 2 shows a preferred embodiment of the pressure application element;

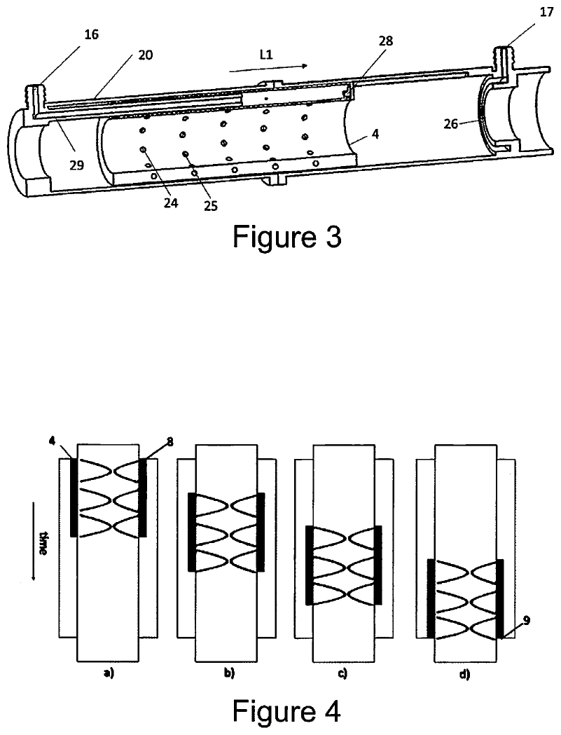

FIG. 3 shows a cross-section in longitudinal direction through the pressure application element;

FIG. 4 illustrates the working of the first preferred embodiment of an implantable oesophagus prosthesis according to the invention;

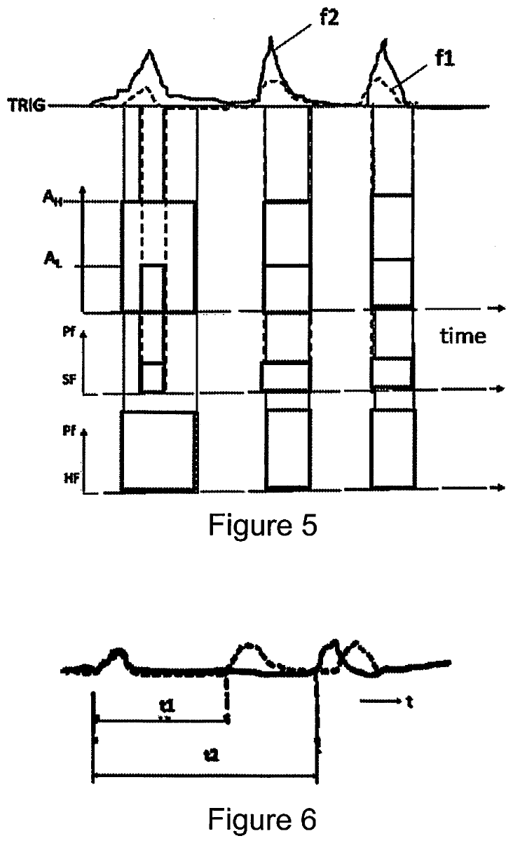

FIG. 5 illustrates the control of the pump;

FIG. 6 shows an example of the second trigger signal;

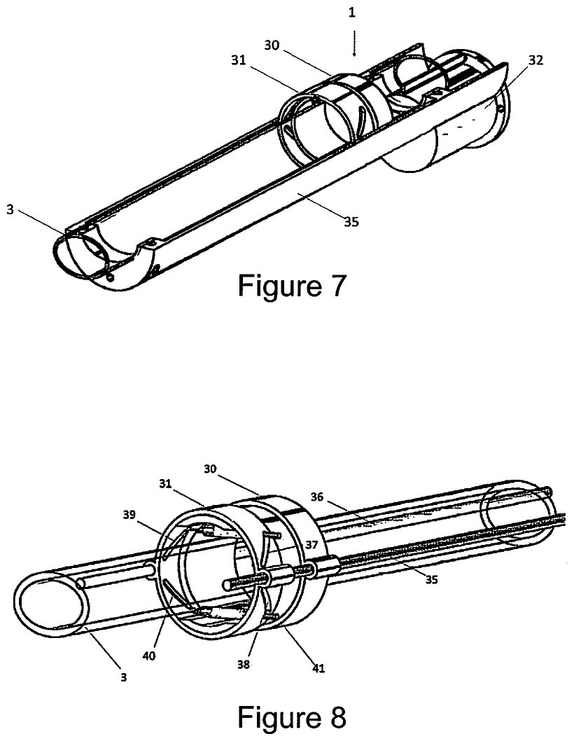

FIG. 7 shows a second embodiment of the implantable oesophagus prosthesis according to the invention;

FIG. 8 illustrates the pressure application element of the second embodiment;

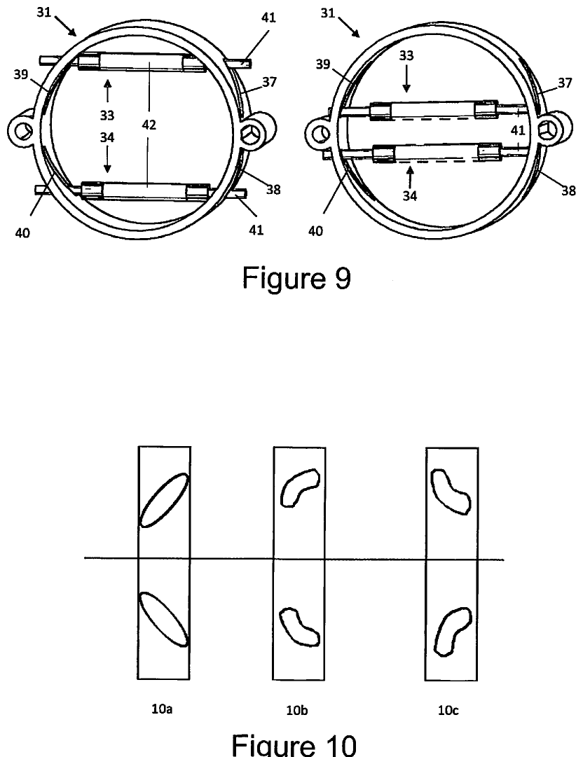

FIG. 9 illustrates the compressor of the second embodiment;

FIG. 10 shows different shapes of the slits;

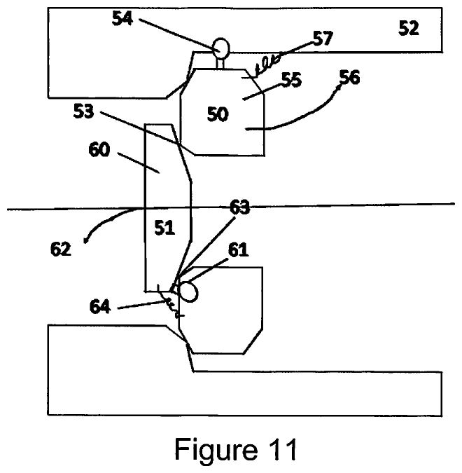

FIG. 11 shows a cross-section through an example of the food passage control valve 7 to be placed in the feeding tube; and

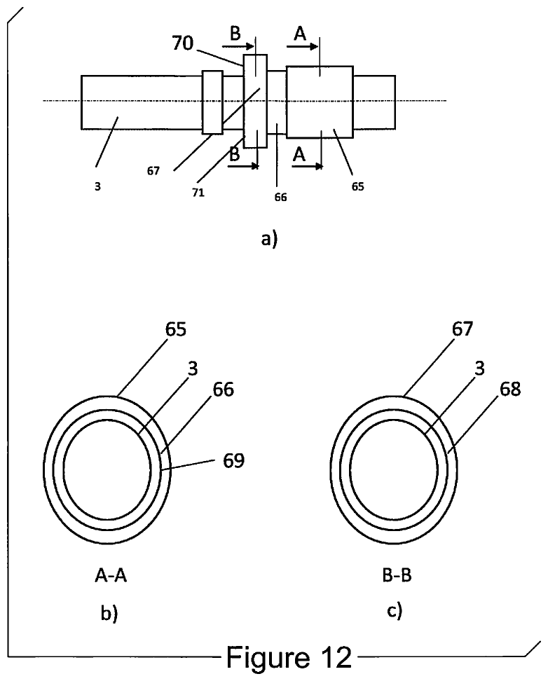

FIG. 12 shows a particular embodiment of the pump.

In the drawings a same reference number has been allotted to a same or analogous element.

FIG. 1 shows a first preferred embodiment of an implantable oesophagus prosthesis 1 according to the invention. The implantable oesophagus prosthesis comprises a feeding tube 3 provided for replacing a natural oesophagus in a human body of a person. The feeding tube is at least partially surrounded by a housing 2 in which a pressurizer member 4,5 is applied in such a manner as to be moved along the tube over a predetermined distance starting from an initial position 8, where the pressurizer member is located in the uppermost part of the housing, as shown in FIG. 1.

The pressurizer member comprises a pressure application element 4 provided for generating a continuous peristaltic pressure against an outer wall of the feeding tube 3 during its movement along the tube in a first direction l1 extending towards a stomach of the human body in which the prosthesis is implanted. The pressurizer member further comprising a return member 5, for example formed by a spring, provided for applying a return force extending in a second direction l2, opposite to the first direction l1, for returning the pressure application element to the initial position 8. In the shown embodiment a compression spring is used. Alternatively, a tension spring could be used, which would than be applied on the upper side of the pressure application element.

The implantable oesophagus prosthesis 1 preferably also comprises a food passage control valve 7 placed at the lowermost part of the housing corresponding with a final position 9 of the pressurizer member. The food passage control valve serves on the one hand to enable the transported food to get access to the stomach. On the other hand, the food passage control valve enables the content of the stomach to flow back via the feeding tube in case of vomiting. The food passage control valve also serves to make a separation between the mouth area of the person and his or her stomach, so that smells from the stomach will not reach the mouth. The valve will open when the food has reached the final position 9 in the feeding tube 3. The opening pressure is preferably set at 25 mbar as it is realised in the human body. As soon as the food has passed the valve, the valve will then close again, for example by means of a returning force applied by a further spring.

The valve will also close the return from the stomach to the mouth so it would not allow the fluid coming from the stomach to return back to the mouth in case the patient has to vomit. This would lead to a very high pressure inside the stomach and potentially to severe damages caused by overpressure. Consequently, the valve has to allow the food to take the opposite direction, this time from the stomach towards the mouth. In this case, the opening pressure must be set to a high value of for example between 150 and 200 mbar. Indeed, because in case of gas present in gaseous drinks or in case of vomiting, high pressures arise and there must be a solid separation between the normal mode and the vomiting mode. A more detailed description of such an anti-reflux and vomit valve will be provided hereinafter.

The implantable oesophagus prosthesis 1 preferably further comprises a food passage detection sensor 10 mounted upstream of the pressure application element 4 on the feeding tube 3 and connected to a control unit 12 and a power unit 11, for example a battery. The control unit is also connected to a pump 14, which is powered by the power unit 11. The pump has an input line connected with a fluid reservoir 13 and an output line connected via a pressure limiting valve 15 to a fluid input 16 of the housing 2. The fluid reservoir is provided for containing a hydraulic fluid such as for example water, or a gas under pressure. The pump is provided for supplying the hydraulic fluid at an overpressure with respect to atmospheric pressure of 0.5 to 2 bar. The housing further comprises a fluid output 17 connected with the fluid reservoir.

The food passage detection sensor 10 is preferably a pressure sensor provided for measuring a pressure exerted on the feeding tube by the food present therein. The food passage detection sensor 10 can also be a detector using a light beam, an ultra-sonic detector or a pressure gauge. The purpose of the food passage detection sensor is to detect that food has entered the feeding tube. When a light beam detector is used it will than emit a light beam and detect the interruption of the light beam by the food. The ultra-sonic detector would emit an ultra-sonic beam and changes in the reflection of this beam caused by the food would be detected. For the sake of clarity, the description will however be limited to a pressure sensor.

FIGS. 2 and 3 shows a preferred embodiment of the pressure application element 4. A partial cut-out is showed in the FIG. 2 so as to show a part of the inner structure of the pressure application element. FIG. 3 shows a cross-section in longitudinal direction through the pressure application element. The pressure application element comprises a body 20, preferably cylindrically shaped, having a fluid input 21 and a fluid output 22. The use of a cylindrically shaped body enables to couple in an appropriate manner with the cylindrical tube, The pressure application member also comprises an inner wall 23 provided with at least a first series of output gates 24 and at least a second series of input gates 25. The input gates being applied, considered in said first direction l1, under the output gates. The output 24, respectively the input 25, gates being connected with the fluid input 21, respectively the fluid output 22. The fluid input 21 cooperates with the fluid input 16 and the fluid output 22 cooperates with the fluid output 17. The first series of output gates 24 comprises at least two output gates and the second series of input gates comprises at least one input gate.

As shown in FIG. 3, a pressure tube 29 is connected to the fluid input 16 and extends in parallel with the inner wall of the body 20. The pressure tube 29 further extends into an inner chamber 28 of the pressure application element 4, which inner chamber starts from the fluid input. This inner chamber 28 has a bottom wall connected with the first series of output gates 24. The input gates are connected with a further chamber (not shown) which is connected with the fluid output 22.

The pressure application element 4 preferably comprises a guiding bar 27 applied on the outer wall of the body 20 and engaged in a guiding channel (not shown) applied on the inner wall of the housing 2. The guiding bar 27 and the guiding channel avoid that the cylindrically shaped body would rotate inside the housing during its movement inside the housing thereby damaging the pressure tube 29.

FIG. 4 illustrates the working of the first preferred embodiment of an implantable oesophagus prosthesis 1 according to the invention. When food reaches an entrance of the feeding tube 3, the pressure sensor 10 detects the pressure applied by the food on the feeding tube. The pressure sensor 10 than generates a first trigger signal which value is dependent from the pressure applied by the food in the feeding tube. The first trigger signal is supplied to the control unit 12 which verifies the value supplied by the first trigger signal. If the latter value exceeds a predetermined threshold value, the control unit generates a second trigger signal which is supplied to the pump 14. Under control of the second trigger signal the pump is activated and starts pumping fluid out of the fluid reservoir 13. The pumped fluid is fed under pressure towards the fluid input 16 and will reach the pressure tube 29.

As the pressure tube 29 extends into the inner chamber 28, the latter will be filled with fluid under pressure which will reach the first set of output gates 24. The fluid under pressure will exhaust by the first set of output gates 24 and will be ejected under pressure against the outer wall of the feeding tube thereby applying a continuous peristaltic pressure against an outer wall of the tube 3, as is illustrated FIG. 4a. In the embodiment show in FIG. 4a, the first set of output gates comprises three subsequent gates which explains why three subsequent wave shape pressure oscillations are shown. It will be clear that the number of three oscillations is only given by way of example and that at least one oscillation would be enough to create a pressure on the outer wall of the feeding tube.

By ejecting the fluid against the outer wall of the feeding tube by means of the first set of output gates, the feeding tube gets locally contracted. Once the targeted compression of the feeding tube is reached, the fluid pressure will cause the moving of the pressure application element along the axis l1 from the initial position 8 to the final position 9, as successively illustrated in the FIGS. 4b, c and d. The pressure applied against the feeding tube and the movement of the pressure application element will cause the food present in the tube to move towards the stomach. This movement also acts against the return member 5.

As long as the pressure application element moves along the axis l1, the compression effect is still active, i.e., the location where the compression takes place is moving together with the pressure application element. This contraction movement leads to the required continuous peristaltic pressure compression wave applied on the feeding tube. Once the pressure application element has reached the final position 9 situated at the bottom of the housing, the pump 14 will stop delivering. The effect is a rapid pressure decay of the applied pressure and so a drop down of the pressure applied against the feeding tube. The effect is that the compression of the feeding tube stops, and the return member will then push the pressure application element back to its initial position 8.

Between two rows of the first set of output gates 24, there is a row of the second set of input gates 25 through which the ejected fluid can be collected after having applied the pressure against the feeding tube in such a manner as to avoid a ‘block compression’ of the feeding tube. The number of input gates sets is preferably one less than the number of sets of output gates. At the level of the space at the initial position 8 and the space at the final position 9 the pressure application element must be connected to the return path 26 so that above and below the back pressure always applies. The fluid collected by the input gates 25 is temporarily stored in the further chamber during the downward movement of the pressure application element and is supplied to the return path 26 when the pressure application member has reached its final position. The return path is connected to the first fluid output so as to enable a flow back of the fluid towards the fluid reservoir 13. In such a manner the fluid is kept in a closed circuit and the prosthesis can work as a stand-alone device within the person in whom it is implanted. Optionally a suction pump can be used to bring the fluid back to the fluid reservoir. Such a suction pump is started when the pressure application member has reached its lowest position in the housing.

In the embodiment shown in FIG. 1, the fluid reservoir is outside the housing, but alternatively the fluid reservoir and/or the pump could also be incorporated in the housing. The same applies for the control unit 12 and the power unit 11. The power unit can also be embodied inside a blister and configured as a plug-in unit which would offer a possibility to easily exchange the power unit when it is empty. Where a plug-in unit is used, care has to be taken that the connection is fluid-tight, and that the unit is sterilised. It is further possible to recharge the battery by induction.

The dimension of the feeding tube 3 will be selected according to the patient morphology. Preferably the feeding tube is manufactured by using a 3D print process, in particular a stereo lithographic process, where a resin-based material is used. The used material should of course be bio-compatible and can for example make use of biological cells. The feeding tube has four connection points to be considered, i.e., two seals 18 and 19 towards the remaining ends of the feeding tube of the person and two connections with the artificial oesophagus housing 2. The feeding tube is preferably realised out of a formed part allowing the extremities realised out of a very soft silicone tube which will allow to be sealed to the remaining feeding tube ends of the patient. At these extremities, there are no special requirements to the roundness of the tube. However, at the connection points to the main body, there are preferably mechanical inlays integrated in the feeding tube which will apply a mechanical pressure at the connection between tube and main body with the intention to seal the feeding tube to the outside. These inlays can be out of metals or any other adapted material to apply stress at the connection point.

The inside of the feeding tube is preferably be formed with a high request on the circularity, in particular for the first embodiment using a pressurized fluid or a gas. Care must be taken that the tube comes back to a round shape, preferably within less than one second, after each compression in order to avoid any mechanical friction with the moving pressurizer. At the same time, the wall thickness of the tube as well as the hardness, have to be well chosen so that they are rigid enough to find back its round shape after compression but still allows the compression with least effort. Tests have shown that a combination of a wall thickness of 2-3 mm and a hardness of 50 Shore A are able to fulfil the needs. Fluid tightness is also a requirement of the prosthesis. This is why, in addition to the mechanical seals, a second type a seal is foreseen, e.g., with glue. An overall flexibility of the prosthesis is further advantageous so as to be able to adjust it to the body morphology of the patient and be able to follow movements imposed on the implanted prosthesis.

Preferably the feeding tube is made by using a material called “Elastic 50A” and commercialised by Fa. Formlabs (https://support.formlabs.com/s/article/Using-Elastic-Resin?language=en US). Elastic 50A resin is an elastomeric material designed for applications requiring high elongation and high energy return. This material is particularly suitable for objects that need to be bend, stretched, compressed, and hold up to repeated cycles without tearing. The material is also transparent, which makes it well suited for medical models for simulation or education. In comparison with other materials the elasticity has the ability to resume its normal shape after being stretched or compressed. Elastic 50A Resin is designed to “bounce back” and return to its original shape quickly. The durometer being the hardness of a material, Elastic 50A Resin has a lower durometer than other Formlabs resins, making it suitable for prototyping parts normally produced with silicone. Elastic 50A Resin also has a higher tear strength than comparable materials, making it capable of repeated cycles of use.

The physical parameters of Elastic 50A are:
Durometer/shore hardness: 50A
Elongation at break (%): 160
Tensile strength (MPa): 3.2
Tear strength (kN/m): 19.1
Colour clear

Moreover the aspect to have it as a translucid material is of importance, too because it allows to use UV active glues for sealings. The material comes as a fluid resin which is then printed in the so-called SLA process (stereolithography).

The pressure to be applied on the feeding tube depends on the nature of the food. If the food is hard, there need to be a high compression of the feeding tube and therefore the control unit 12 will request a high delivery rate to the pump. This is translated into the pump speed which is equivalent to the pump flow and is illustrated in FIG. 5. In FIG. 5 the curve f1, respectively f2, shows the value of the first trigger signal generated by the pressure sensor 10 in case of detection of soft, respectively hard, food. The amplitude of the signal in curve f1 is lower as the one in curve f2, as a lower pressure was detected for soft food. Also, the time during which the pressure sensor will detect the pressure applied by the food is taken into account upon generating the first trigger signal. The first trigger signal will than be used by the control unit to determine the amplitude and the period of the second trigger signal supplied to the pump. As illustrated in FIG. 5 the amplitude and period of the second trigger signal AL, generated when soft food (SF) is detected, is lower and shorter than the one of AH when harder food (HF) is detected.

The control unit is also provided for analysing the swallowing frequency, because patients will eat at different speed, so for example some patients will eat slowly, whereas others will eat fast. This swallowing frequency is determined by considering two subsequent events when the sensor signal f1 and f2 amplitude exceeds the trigger level. This characteristic time is shown as t1 and t2 in FIG. 6. The pump will be driven with sets of activate and passive pump phases. If the patient is a slow eater, the time between two of these will be larger. The faster the patient is eating, the closer will be the set of these events.

The patient's doctor might be interested to analyse the functioning of the oesophagus prosthesis implanted in the patient as well as the patient swallowing behaviour for an adapted treatment. Therefore, the implantable oesophagus will be preferably equipped with means that allows a wireless transmission of the required data. In the latter case the control unit will be equipped with a memory for storing measured data and provided with a WIFI, Bluetooth or equivalent transmission unit in order to read the stored data.

It could also be of interest to set or adapt the setting of certain operation parameters from the outside. To this purpose the implantable oesophagus prosthesis preferably comprises an interface provided to communicate with the control unit so as to set parameters from the outside which can differ from those which would be set upon initialisation of the prosthesis.

It can also be considered to mount other sensors in the prosthesis. Those sensors will the of course be connected to the control unit. Thus an accelerometer could be integrated for detecting the movements of the patient in whom the prosthesis is implanted. Thus the management of the applied peristaltic pressure can take into account the movements of the patient. Instead of an accelerometer, or even in combination with an accelerometer, one could consider to foresee a gyroscope which would enable to detect in which position, sitting down or standing up, the patient is. The mounting of a temperature sensor and/or a humidity sensor could also be considered.

FIG. 12a shows a particular embodiment of the pump 14. FIG. 12b, respectively 12c, shows a cross-section along the line A-A, respectively B-B, through the pump. In this embodiment the pump is as if to say wrapped around the feeding tube 3. The pump comprises an input 70 which will be connected to the fluid reservoir 13 as well as an output 71 also connected to the fluid reservoir. The pump comprises a stator 65 which is part of an electrical engine driving the pump. The stator is fixed and wrapped around a rotor 66, as illustrated in FIG. 12b. The rotor is equipped on its internal peripheral with at least one protrusion 69, which is in contact with an internal ring 68, which is part of the pumping element of the pump. The internal ring is wrapped around an external ring 67, which is fixed. When the pump is activated the rotor 66 will be driven in rotation inside the stator 65. The rotation of the rotor will on its turn drive the protrusion 69 which will exert a pumping action on the internal ring 67 for pumping fluid. As the pump is wrapped around the feeding tube, the fluid thus pumped will be directly furnished to the fluid input 16. The fact that the pump is wrapped around the feeding tube has for advantage to save space. This is of particular interest in case where an implanted oesophageal has to be implanted in a human body in which the available space is very limited.

It will be noted that such an embodiment of a pump is not limited to an application in an implanted oesophageal implant and can be applied to other devices such as electrical household appliances, medical pumps, and so on.

According to a second preferred embodiment of an implantable oesophagus prosthesis according to the invention and shown in the FIGS. 7 to 10, the pressure application element comprises a pusher 30 and a compressor 31 mounted on a driving member 32 provided for enabling the up and downward movement along the feeding tube 3. The compressor 31 is mounted downstream of the pusher 30 considered in the direction along which l1 extends. The compressor comprises a set of compression members 33, 34 provided for exerting a back-and-forth movement with respect to the feeding tube 3. The pusher being provided for imposing a movement on the compression elements in order to enable said back and forth movement.

The driving element 32 comprises an electrical engine connected to a compressor rod 36 and a pusher rod 35. The compressor rod 36 is connected to the compressor 31 and the pusher rod 35 is connected to the pusher 30, as illustrated in FIG. 8. As illustrated in FIGS. 9a and 9b, the compressor 31 comprises a ring provided with a set of slits 37, 38, 39 and 40 which are applied so as to be tilted with respect to the first direction l1 in which the pressure application element moves. The slits 37 and 38, respectively 39 and 40, are applied so as to form a V shape with respect to a middle axis of the ring. The axis 41 of the compression member 33, respectively 34, is applied in the slits 37 and 39, respectively 38 and 40 so as to be moved inside and guided by the slits in which the axis is applied. The slits 37 and 39, respectively 38 and 40, are applied on opposite sides of the ring with respect to each other.

In FIG. 7 and FIG. 10a the slits extend linearly over their length. It is however also possible that the slits extend in a curved shape as shown in FIG. 10b, or in a concave shape as shown in FIG. 10c. The shape of the slits will have an effect on the pressure applied on the feeding tube and on the energy consumption. The shape of the slits will have an impact on the torque profile applied by the driving member and thus on the energy consumption.

At the beginning of the compression process the compressor 31 and the pusher 30 are separated by a predetermined distance, as shown in FIG. 7, which allows the compression members to be placed on the upper, respectively lower end of the tilted slits as illustrated in FIG. 9a. In this position no compression is applied on the feeding tube. During an initial operational phase, the electrical engine of the driving member 32 starts to rotate the compressor rod 36 in counter clockwise rotation, whereas the pusher rod 35 is not driven. This will cause the pusher 30 to move towards the compressor 31. During a first operational phase, starting once the pusher has reached the compressor, the pusher rod will push on the axis on which the compression members are mounted and will cause them to be moved inside the tilted slit for imposing a forth movement on the compression members.

By doing this the axis, on which the compression members are mounted, moves in the slits towards the centre of the compressor, as shown in FIG. 9b, and compresses the feeding tube. Once the compression is completed, a second operational phase is started during which the driving member turn clockwise and move both the pusher and the compressor along the feeding tube in the direction l1, while keeping the feeding tube under compression for realising in such a manner a continuous peristaltic pressure against the wall of the feeding tube. At the end of this second operational phase, a third operational phase is started during which the compressor rod turns clockwise whereas the pusher rod is standing still. The effect is that the compressor and the pusher separate and that the compressor members will move back to their initial position in the slits. Consequently, the feeding tube gets uncompressed. Once the compression completely released, a fourth operational phase is started during which the driving member, which than plays the role of the return member, will impose a counter clockwise movement on compressor and the pusher for returning them back to the initial upper position. The third and fourth operational phase are either subsequent to each other, or coincident in time with each other.

FIG. 11 shows transversal cross-section through an example of the food passage control valve 7 to be placed in the feeding tube downstream of the final position 9 reached by the pressure application element upon its movement along the feeding tube. The food passage control valve 7 comprises a first 50 and a second 51 valve element. The first valve element 50 is co-axially applied with respect to the second valve element 51. The first valve element being provided to allow a first passage from the tube to the stomach, whereas the second valve element being provided to allow a second passage from the stomach to the tube. Preferably the first and second valve element extend in a same horizontal plane.

FIG. 11 further shows that the first valve element 50 is housed in a valve body 52 provided with a valve seat 53 formed by a cavity applied in the valve body. The valve seat is preferably conically shaped for enabling a more precise opening pressure. The first valve element comprises a first articulation element 54 pivotably applied in the valve seat. A first valve plate 55 is connected to the first articulation element 54 and so pivotable in the feeding tube in a direction towards the stomach as indicated by the arrow 56. A torsion spring 57 is applied between the first valve plate and the valve body for enabling a return of the first valve plate towards an initial position where the first valve element is in a closed position. The first valve element is preferably calibrated at an opening pressure of approximately 25 mbar. The first valve plate completely closes the feeding tube so as to avoid a return from the stomach towards the patient's mouth.

The second valve element 51 comprises a second valve plate 60 applied on the first valve plate 55. The second valve element comprises a second articulation element 61 pivotably applied in the first valve element. A second valve plate 60 is pivotable in the feeding tube in a direction from the stomach, as indicated by the arrow 62. The valve seat 63 in which the second articulation element is lodged is preferably conically shaped. The opening pressure of the second valve plate is preferably calibrated at a pressure of at least 150 mbar, preferably 200 mbar. A further torsion spring 64 is applied between the second valve plate and the first valve plate for enabling a return of the second valve plate towards an initial position where the second valve element is in a closed position.

It should be noted that the French patent 2 621 813 describes a valve provided for being implanted in an implantable oesophageal prosthesis. However the known valve does not has an opening pressure difference depending on the opening direction.

It should be noted that passage control valve shown in FIG. 11 is not only suitable to be used in an implantable oesophagus prosthesis and can also be used as a general purpose safety valve in conducts or devices where a fluid, a gas or even solid materials are circulating. The general purpose safety valve can be used in applications where a bi-directional flow has to be controlled, such as for example in oil-pumping, gas-pumping, streams of chemical products or food. Of course the pressure at which the valve will open will depend on the field in which the valve is used.

1. Implantable oesophageal prosthesis (1) comprising a feeding tube (3), which feeding tube is at least partially surrounded by a housing (2) in which a pressurising member (4,5) is applied, which pressurising member comprises a pressure application element (4) provided for generating a peristaltic pressure against an outer wall of the feeding tube, characterised in that the pressurising member is applied in such a manner as to be movable along the feeding tube over a predetermined distance starting from an initial position (8), which pressure application element (4) is provided for generating the peristaltic pressure continuously during its movement along the feeding tube in a first direction (l1) extending towards the stomach of a body in which the prosthesis is to be implanted, said pressurising member comprises a return member (5) provided for applying a return force extending in a second direction (l2), opposite to the first direction, in order to return the pressure application element towards its initial position.

2. Implantable oesophageal prosthesis as claimed in claim 1, characterised in that the pressure application element (4) is provided for generating the continuous peristaltic pressure by using a hydraulic fluid or a gas under pressure.

3. Implantable oesophageal prosthesis as claimed in claim 2, characterised in that the pressure application element (4) comprises a fluid input (16) and a fluid output (17), which the pressure application element comprises an internal wall configured for at least partially enveloping the feeding tube and provided with a first series of output gates (24) and at least a second series of input gates (25), the input gates being applied, considered in said first direction, under the output gates, the output gates, respectively the input gates, being connected to said fluid input (16), respectively to said fluid output (17), the fluid input being connected to an pump output (14) provided for furnishing the hydraulic fluid at a pressure of at least 2 bars.

4. Implantable oesophageal prosthesis as claimed in claim 3, characterised in that the first series of output gates (24) comprises at least two output gates and the second series of input gates (25) comprises at least one input gate.

5. Implantable oesophageal prosthesis as claimed in claim 3, characterised in that it comprises a pressure sensor (10) provided for measuring a pressure applied by food present in the feeding tube, said prosthesis also comprises a control unit (12) connected to said pressure sensor, the pressure sensor being provided for generating a first trigger signal on the basis of the measured pressure, said control unit being provided for generating, on the basis of said first trigger signal, a second trigger signal having an amplitude and a period to be furnished to the pump (14) for controlling the latter.

6. Implantable oesophageal prosthesis as claimed in claim 3, characterised in that the pump (14) is wrapped around the feeding tube (3).

7. Implantable oesophageal prosthesis as claimed in claim 1, characterised in that the return member (5) is formed by a spring lodged at the inside of said housing.

8. Implantable oesophageal prosthesis as claimed in claim 1, characterised in that the pressure application element comprises a set of compression members (33,34) provided for exerting a back and forth movement with respect to the feeding tube (3).

9. Implantable oesophageal prosthesis as claimed in claim 8, characterised in that the pressure application element comprises a pusher (30) and a compressor (31) mounted on a driving member (32) provided for generating said movement along the tube, said compressor being mounted downstream of said pusher, said pressure members being part of said compressor, said pusher being provided for imposing the back and forth movement to said compression members.

10. Implantable oesophageal prosthesis as claimed in claim 9, characterised in that said driving member is provided for driving the pusher during a first operational phase for imposing said forth movement on said compression members and for, during a second operational phase, following the first operational phase, imposing said movement along the tube in said first direction l1.

11. Implantable oesophageal prosthesis as claimed in claim 10, characterised in that said driving member is provided for during a third operational phase, following said second operational phase, driving the pusher for imposing said back movement on said compression members and for during a fourth operational phase imposing said return movement along the feeding tube along said second direction l2.

12. Implantable oesophageal prosthesis as claimed in claim 11, characterised in that said third and fourth operational phases are either subsequent to each other, or coincident in time with each other

13. Implantable oesophageal prosthesis as claimed in claim 8, characterised in that the compression members are each formed by a roll applied in rotation on an axis (41).

14. Implantable oesophageal prosthesis as claimed in claim 13, characterised in that the compressor (31) comprises a ring provided with a set of splits (37,38,39,40) which are applied in such a manner as to be inclined with respect to the first direction, said set of splits comprising two splits per compression element applied on opposite sides of said ring for receiving said axis.

15. Implantable oesophageal prosthesis as claimed in claim 1, characterised in that it comprises a food passage control valve (7) applied in said feeding tube downwards a final position (9) reached by the pressure application element during its movement along the feeding tube.

16. Implantable oesophageal prosthesis as claimed in claim 15, characterised in that the food passage control valve (7) comprises a first and a second valve element, the first valve element being coaxially applied with respect to the second valve element, the first valve element being provided for enabling a first passage of the feeding tube to the stomach, the second valve element being provided for enabling a second passage from the stomach to the feeding tube.

17. Implantable oesophageal prosthesis as claimed in claim 16, characterised in that the first, respectively the second valve element is calibrated at an opening pressure of about 25 mbar, respectively an opening pressure of at least 150 mbar.

18. Implantable oesophageal prosthesis as claimed in claim 4, characterised in that it comprises a pressure sensor (10) provided for measuring a pressure applied by food present in the feeding tube, said prosthesis also comprises a control unit (12) connected to said pressure sensor, the pressure sensor being provided for generating a first trigger signal on the basis of the measured pressure, said control unit being provided for generating, on the basis of said first trigger signal, a second trigger signal having an amplitude and a period to be furnished to the pump (14) for controlling the latter.

19. Implantable oesophageal prosthesis as claimed in claim 4, characterised in that the pump (14) is wrapped around the feeding tube (3).

20. Implantable oesophageal prosthesis as claimed in claim 16, characterised in that the first, respectively the second valve element is calibrated at an opening pressure of about 25 mbar, respectively an opening pressure of at least 200 mbar.
